(19) United States
(12) Patent Application Publication
(10) Pub. No.: US 2022/0401014 A1
(43) Pub. Date: Dec. 22, 2022

(54) PORTABLE SKIN CONDITION MEASURING APPARATUS, SKIN CONDITION CARE SYSTEM INCLUDING THE SAME, AND SKIN CONDITION CARE METHOD

(71) Applicant: GPOWER INC., Gyeonggi-do (KR)

(72) Inventors: Chang Hee HAN, Gyeonggi-do (KR); Deug Ki LEE, Seoul (KR)

(21) Appl. No.: 17/845,151

(22) Filed: Jun. 21, 2022

(30) Foreign Application Priority Data
Jun. 21, 2021 (KR) 10-2021-0080316

(51) Int. Cl. A61B 5/00; G16H 30/20

(52) U.S. Cl. CPC A61B 5/442; G16H 30/20; A61B 5/0077; A61B 2576/02

(57) ABSTRACT

A portable skin condition measuring apparatus according to an embodiment of the present disclosure is capable of complexly measuring and analyzing various skin condition indexes of a user by coming into contact with a user's face, body, or scalp. The portable skin condition measuring apparatus may solve inconvenience in measuring a user's skin condition and improve efficiency of measurement, determination, and care of the user's skin condition by measuring and analyzing various skin condition indexes of the user in a non-face-to-face manner.

### CROSS-REFERENCE TO RELATED APPLICATION AND CLAIM OF PRIORITY

This application claims the benefit under 35 U.S.C. § 119 of Korean Patent Application No. 10-2021-0080316, filed on Jun. 21, 2021, in the Korean Intellectual Property Office, the entire disclosure of which is incorporated herein by reference for all purposes.

### BACKGROUND

### 1. Field

The present disclosure relates to a skin condition measuring apparatus, and more particularly, to a portable skin condition measuring apparatus capable of complexly measuring and analyzing various skin condition indexes of a user, a skin condition care system including the same, and a skin condition care method.

### 2. Description of Related Art

As people's interest in beauty increases, interest in skin beauty has also increased. According to such a trend, various skin condition measuring apparatuses capable of measuring and analyzing skin conditions of people have been developed and commercialized.

Existing skin condition measuring apparatuses include a skin barrier index measuring apparatus, a skin beauty index measuring apparatus, a skin aging index measuring apparatus, and the like. The skin barrier index measuring apparatus is an apparatus that measures a level of a skin barrier function by sensing a stratum corneum hydration or a transepidermal water loss. In addition, the skin beauty index measuring apparatus and the skin aging index measuring apparatus are apparatuses that measure skin conditions such as pores, sebum, wrinkles, and pigments by analyzing an image obtained by capturing an image of a predetermined skin region.

Such existing skin condition measuring apparatuses are possessed in specific places, for example, hospitals for skin treatment or companies for skin care. Accordingly, people directly visit the hospitals or the companies, measure their skin conditions using the skin condition measuring apparatuses possessed by the hospitals or the companies, and get skin care according to measurement results.

In addition, each of the existing skin condition measuring apparatuses includes expensive individual apparatuses, and accordingly, people should use a plurality of apparatuses in order to measure their skin barrier indexes, skin beauty indexes, or skin aging indexes, which was inconvenient.

Moreover, it has become difficult to visit the hospitals or the companies in order to measure the skin conditions at a time when non-face-to-face between people is considered important due to the corona virus, such that inconvenience of people who want to get the skin care has increased.

### SUMMARY

An object of the present disclosure is to provide a portable skin condition measuring apparatus capable of complexly measuring and analyzing various skin condition indexes of a user, a skin condition care system including the same, and a skin condition care method.

According to an embodiment of the present disclosure, a portable skin condition measuring apparatus includes: a main body having a skin measurement region formed therein; a sensor unit disposed inside the main body and measuring a user's skin condition through the skin measurement region; an image capturing unit disposed inside the main body and capturing an image of a user's skin through the skin measurement region; and a control unit controlling an operation of at least one of the sensor unit and the image capturing unit based on a control signal transmitted from a user terminal, and transmitting at least one of measurement results of the sensor unit and a skin image of the image capturing unit to the user terminal.

Here, the skin measurement region of the main body is partitioned into a first region and a second region by a partitioning part, and the first region has a shape in which one surface thereof in contact with the user's skin is opened.

According to another embodiment of the present disclosure, a skin condition care system includes: the skin condition measuring apparatus described above; a user terminal controlling an operation of the skin condition measuring apparatus based on one or more measurement modes selected among a plurality of skin measurement modes, receiving at least one of a transepidermal water loss amount, a skin water content, and a skin image for the user's skin, and analyzing the user's skin condition; and a skin care server receiving an analysis result from the user terminal, determining the user's skin condition, and storing a determination result.

The skin measurement mode includes a transepidermal water loss measurement mode, a stratum corneum hydration measurement mode, or a skin image measurement mode.

According to still another embodiment of the present disclosure, a skin condition care method includes: generating a control signal for controlling an operation of the skin condition measuring apparatus based on at least one of a transepidermal water loss measurement mode, a stratum corneum hydration measurement mode, or a skin image measurement mode selected through the user terminal; measuring a user's skin condition through the skin condition measuring apparatus controlled by the control signal; receiving at least one of measurement results and a skin image from the skin condition measuring apparatus and analyzing the user's skin condition; and determining the user's skin condition by synthesizing the analysis results, and storing a determination result.

### BRIEF DESCRIPTION OF THE DRAWINGS

FIG. 1 is a view illustrating a skin condition care system according to an embodiment of the present disclosure.

FIG. 2 is a view illustrating a portable skin condition measuring apparatus according to an embodiment of the present disclosure.

FIG. 3 is a block diagram illustrating components of the skin condition measuring apparatus of FIG. 2.

FIG. 4 is a cross-sectional view illustrating a cross section of the skin condition measuring apparatus of FIG. 2.

FIG. 5 is a view illustrating a first sensor part of FIG. 4.

FIG. 6 is a view illustrating a skin condition care method according to an embodiment of the present disclosure.

### DETAILED DESCRIPTION OF THE EMBODIMENTS

General terms have been selected in consideration of functions in various embodiments of the present disclosure as terms used in the present specification and the claims. However, these terms may be changed depending on an intention of those skilled in the art, legal or technical interpretation, the emergence of a new technology, and the like. In addition, some terms may be terms arbitrarily selected by an applicant. These terms may be interpreted in the meanings defined in the present specification, and may be interpreted based on a general content of the specification and usual technical knowledge in the art as long as they are not specifically defined.

In addition, throughout the accompanying drawings of the present specification, the same reference numerals denote parts or components performing substantially the same functions. For convenience of explanation and understanding, different embodiments will be described using the same reference numerals. That is, even though all the components having the same reference numerals are illustrated in a plurality of drawings, the plurality of drawings do not mean one embodiment.

In addition, in the present specification and the claims, terms including ordinal numbers such as ‘first’, ‘second’, and the like may be used to distinguish between components. These ordinal numbers are used to distinguish the same or similar components from each other, and the meaning of the terms should not be restrictively construed by the use of these ordinal numbers. As an example, components combined with these ordinal numbers should not be limited in order of use or arrangement by the ordinal numbers. If necessary, the respectively ordinal numbers may be interchangeably used.

In the present specification, singular forms include plural forms unless the context clearly indicates otherwise. It should be understood that terms ‘comprise’ or ‘include’ used in the present specification, specify the presence of features, numerals, steps, operations, components, parts mentioned in the present specification, or combinations thereof, and do not preclude the presence or addition of one or more other features, numerals, steps, operations, components, parts, or combinations thereof.

In addition, in an embodiment of the present disclosure, a case where any portion is referred to as being “connected to” another portion not only includes a case where any one portion and another portion are “directly connected to” each other, but also includes a case where any one portion and another portion are “indirectly connected to” each other through the other medium. In addition, unless explicitly described otherwise, “comprising” any components will be understood to imply the inclusion of other components but not the exclusion of any other components.

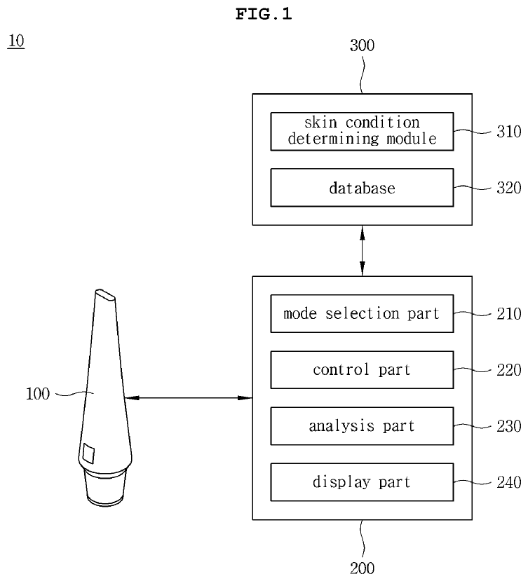

FIG. 1 is a view illustrating a skin condition care system according to an embodiment of the present disclosure.

Referring to FIG. 1, a skin condition care system 10 according to the present embodiment may receive measurement results of a user's skin condition through a skin condition measuring apparatus 100 capable of measuring various skin condition indexes, for example, a skin barrier index, a skin beauty index, or a skin aging index, and analyze and manage the user's skin condition from the measurement results.

The skin condition care system 10 includes a user terminal 200 possessed by each of a plurality of users, a skin condition measuring apparatus 100 matched and connected to the user terminal 200 in a one-to-one manner, and a skin care server 300 connected to the user terminal 200.

The skin condition measuring apparatus 100 is an apparatus that each of a plurality of users possesses in order to measure high/her skin condition, and may measure at least one of various skin conditions of the user, that is, a skin barrier condition, an aging skin condition, or a skin beauty condition according to the control of the user terminal 200.

Such a skin condition measuring apparatus 100 may be configured as a portable apparatus that the user may grip and use with his or her hand, and accordingly, may measure the user's skin condition regardless of time and place.

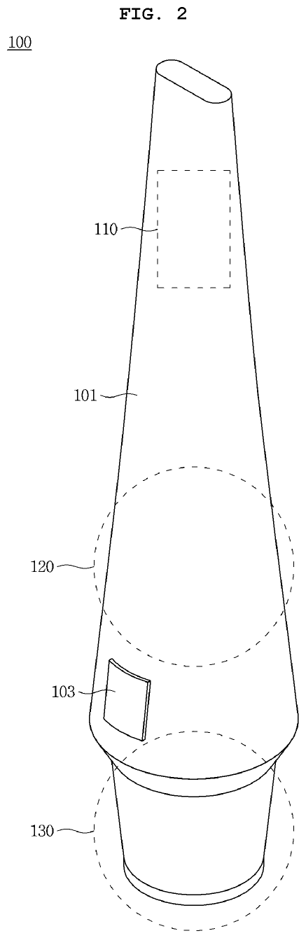

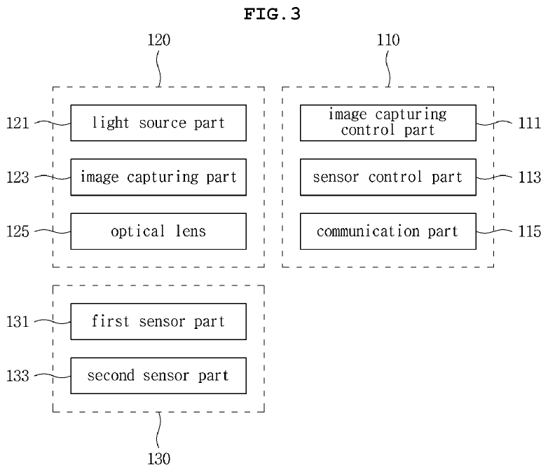

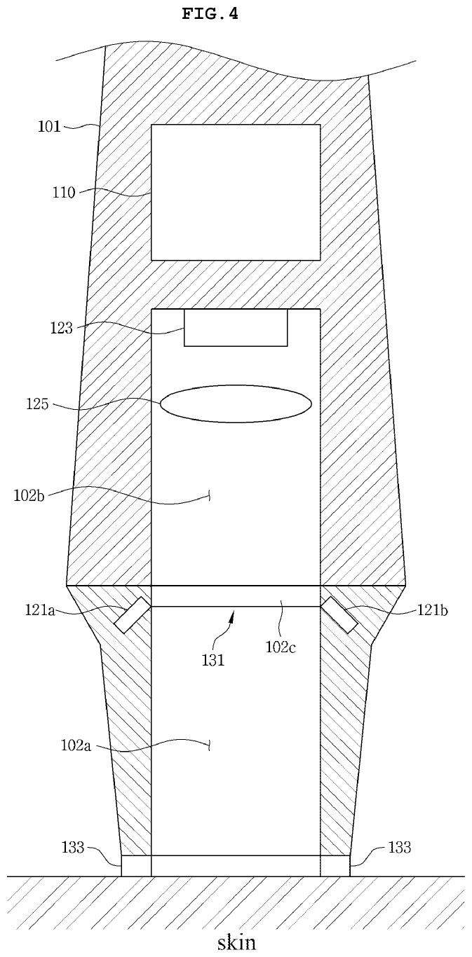

FIG. 2 is a view illustrating a portable skin condition measuring apparatus according to an embodiment of the present disclosure, FIG. 3 is a block diagram illustrating components of the skin condition measuring apparatus of FIG. 2, and FIG. 4 is a cross-sectional view illustrating a cross section of the skin condition measuring apparatus of FIG. 2.

Referring to FIGS. 2 to 4, the skin condition measuring apparatus 100 according to the present embodiment may include a main body 101 and a sensor unit 130, an image capturing unit 120, and a control unit 110 that are disposed inside the main body 101.

The main body 101 may be configured in a hollow shape in which one or more spaces, for example, skin measurement regions 102a and 102b are formed. The skin measurement regions 102a and 102b inside the main body 101 may be formed along a length direction of the skin condition measuring apparatus 100 in a shape in which one surface, for example, one end of the skin condition measuring apparatus 100 in contact with the user's skin is opened.

The skin measurement regions 102a and 102b may include a first region 102a and a second region 102b partitioned by a partitioning part 102c. The first region 102a and the second region 102b may be disposed on the same line and be formed to extend in the length direction of the main body 101.

A sensor unit 130 to be described later may be disposed in the first region 102a and measure the user's skin condition. The image capturing unit 120 maybe disposed in the second region 102b and measure the user's skin condition.

The partitioning part 102c may be disposed in a width direction of the skin measurement regions 102a and 102b, that is, in a direction crossing the skin measurement regions 102a and 102b and partition the first region 102a and the second region 102b. The partitioning part 102c may be transparently formed so that light may pass therethrough for a smooth operation of an image capturing unit 120 to be described later, for example, for capturing an image of the user's skin.

In addition, a power button 103 capable of turning on/off a power supply of the skin condition measuring apparatus 100 may be exposed and disposed on an outer peripheral surface of the main body 101.

The sensor unit 130 may be disposed in the first region 102a of the skin measurement regions 102a and 102b and measure the user's skin condition, for example, the user's skin water condition. The sensor unit 130 may transmit measurement results to the control unit 110. The sensor unit 130 may include a first sensor part 131 and a second sensor part 133.

The first sensor part 131 may be disposed inside the first region 102a and measure an amount of water evaporated and lost from the user's skin, that is, a transepidermal water loss (TEWL).

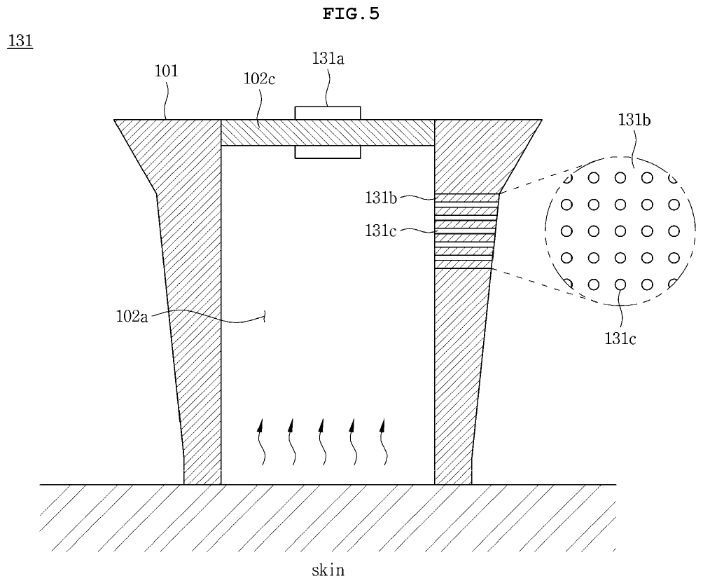

FIG. 5 is a view illustrating a first sensor part of FIG. 4.

Referring to FIG. 5, the first sensor part 131 may include a pressure exhaust adjusting part 131b formed on one sidewall of the first region 102a, for example, one sidewall of the main body 101, and a water amount measuring part 131a disposed on the partitioning part 102c.

The pressure exhaust adjusting part 131b may adjust an internal pressure and a density gradient of the first region 102a. The pressure exhaust adjusting part 131b may be formed on a part of one sidewall of the first region 102a so as to have the same thickness as the one sidewall.

One or more exhaust holes 131c having an inner diameter of 1/100 of a surface area of the first region 102a or a surface area of the pressure exhaust adjusting part 131b may be regularly or irregularly formed in the pressure exhaust adjusting part 131b. The exhaust hole 131c may be formed to have an inner diameter smaller than one side surface of the first region 102a in contact with the skin.

Due to such exhaust holes 131c, in the first region 102a, an internal pressure and a density gradient of the first region 102a according to differences in pressure and density between the inside and the outside of the first region 102a may be adjusted.

For example, an increase rate of a partial pressure of water vapor by evaporation of water from the skin and a transfer rate of a pressure inside the first region 102a may be faster than diffusion and convection by temperature. Accordingly, the internal pressure of the first region 102a may be constantly maintained by exhausting air inside the first region 102a to the outside of the first region 102a through the exhaust holes 131c described above.

The water amount measuring part 131a may measure a transepidermal water loss amount for the user's skin based on a water amount discharged to the outside of the first region 102a through the exhaust holes 131c.

For example, assuming that a temperature increasing during the measurement of the transepidermal water loss amount of the skin is 1° C., a volume increment and a pressure increment may be 0.33%, and the air inside the first region 102a may be exhausted through the exhaust holes 131c of the pressure exhaust adjusting part 131b described above, such that the internal pressure of the first region 102a maybe constantly maintained.

Therefore, the internal pressure of the first region 102a becomes always higher than external pressure, such that the air existing in the first region 102a and including water may be discharged to the outside through the exhaust holes 131c. In this case, since the exhaust holes 131c are in a state in which a diameter and a size thereof are set, the water amount measuring part 131a may measure the transepidermal water loss amount of the user's skin by detecting a water level and a water level change amount in the first region 102a.

The water amount measuring part 131a may transmit the measured transepidermal water loss amount to the control unit 110. Such a water amount measuring part 131a may include one or more humidity sensors or water detection sensors.

Referring to FIGS. 2 to 4 again, one or more second sensor parts 133 may be disposed outside the first region 102a, for example, at one end of the main body 101. The second sensor part 133 may be in contact with the user's skin to measure a water content of the skin, that is, a stratum corneum hydration (SCH).

The image capturing unit 120 maybe disposed in the first region 102a and the second region 102b of the main body 101, capture an image of the user's skin, and transmit the captured skin image to the control unit 110.

The image capturing unit 120 may include a light source part 121 irradiating the user's skin with light through the first region 102a of the main body 101, an image capturing part 123 disposed in the second region 102b of the main body 101 and capturing an image of the user's skin through the first region 102a and the second region 102b, and an optical lens 125 disposed between the image capturing part 123 and the skin in the second region 102b.

The light source part 121 may include a first light source 121a and a second light source 121b disposed to be inclined and symmetrical to each other.

Here, the first light source 121a and the second light source 121b may be disposed to be inclined at an angle of approximately 45° and irradiate to the user's skin with light through the first region 102a. To this end, a light transmitting surface may be formed on an inner surface of the first region 102a so that the light emitted from the first light source 121a and the second light source 121b is transmitted.

In addition, each of the first light source 121a and the second light source 121b may include a plurality of light source elements for emitting white light, ultraviolet light, or polarized light.

For example, each of the first light source 121a and the second light source 121b may include two white light sources and one ultraviolet light source. In addition, each of the first light source 121a and the second light source 121b may include a polarizing filter (not illustrated) disposed on a light emission surface of one of the two white light sources.

Accordingly, the light source part 121 may irradiate the user's skin with white light, ultraviolet light, or polarized light from the first light source 121a and the second light source 121b through the first region 102a according to the control of the control unit 110.

The image capturing part 123 may capture an image of one region of the user's skin irradiated with the light from the light source part 121. The image capturing part 123 may transmit a skin image according to an image capturing result to the control unit 110. Such an image capturing part 123 may be composed of a charge coupled device (CCD) complementary metal oxide semiconductor (CMOS) image sensor, and a spaced distance from the image capturing part 123 to the user's skin may be about 40 mm or less.

The optical lens 125 may be disposed between the image capturing part 123 and the user's skin in the second region 102b. The optical lens 125 may focus light reflected from the skin on the image capturing part 123 when the image capturing part 123 captures an image of the user's skin.

The control unit 110 may control operations of the sensor unit 130 and the image capturing unit 120 described above according to a control signal transmitted from the user terminal 200 to allow the sensor unit 130 and the image capturing unit 120 to measure the user's skin condition. In addition, the control unit 110 may receive measurement results of the skin condition by the sensor unit 130 and the image capturing unit 120, for example, a transepidermal water loss amount, a skin water content, and a skin image, and transmit the measurement results to the user terminal 200. Such a control unit 110 may include an image capturing control part 111, a sensor control part 113, and a communication part 115.

The image capturing control part 111 may control operations of the light source part 121 and the image capturing part 123 of the image capturing unit 120 according to a control signal of the user terminal 200.

For example, the image capturing control part 111 may control the light source part 121 to irradiate the user's skin with the white light, and accordingly, control the image capturing part 123 to capture an image of a surface of the user's skin irradiated with the white light. In addition, the image capturing control part 111 may control the light source part 121 to irradiate the user's skin with the polarized light, and accordingly, control the image capturing part 123 to capture an image of the surface of the user's skin irradiated with the polarized light. In addition, the image capturing control part 111 may control the light source part 121 to irradiate the user's skin with the ultraviolet light, and control the image capturing part 123 to capture an image of the surface of the user's skin irradiated with the ultraviolet light.

The image capturing control part 111 may control an operation of the image capturing unit 120 so that the image capturing unit 120 performs one operation, for example, the image capturing part 123 performs one image capturing by irradiating the user's skin with one of the white light, the polarized light, and the ultraviolet light from the light source part 121. In addition, the image capturing control part 111 may control an operation of the image capturing unit 120 so that the light source part 121 sequentially irradiates the user's skin with the white light, the polarized light, and the ultraviolet light or repeatedly irradiates the user's skin with one of the white light, the polarized light, and the ultraviolet light and the image capturing part 123 performs image capturing whenever the user's skin is irradiated with each light.

The sensor control part 113 may control operations of the first sensor part 131 and the second sensor part 133 of the sensor unit 130 according to a control signal of the user terminal 200.

For example, the sensor control part 113 may perform control so that one of the first sensor part 131 and the second sensor part 133 is operated to measure the user's skin condition or both the first sensor part 131 and the second sensor part 133 are operated to measure the user's skin condition.

In addition, the sensor control part 113 may perform control so that the user's skin condition is measured through the sensor unit 130 together with the operation of the image capturing unit 120 by the image capturing control part 111 or the user's skin condition is measured through the sensor unit 130 before/after the operation of the image capturing unit 120.

The communication part 115 may provide the control signal transmitted from the user terminal 200 to the image capturing control part 111 and the sensor control part 113. In addition, the communication part 115 may transmit the measurement result transmitted from the sensor unit 130 to the user terminal 200 or transmit the skin image transmitted from the image capturing unit 120 to the user terminal 200.

Referring to FIG. 1 again, the user terminal 200 may be connected to the skin condition measuring apparatus 100 through a wireless network (not illustrated) such as wireless fidelity (Wi-Fi) or Bluetooth. Such a user terminal 200 may be a smartphone or a tablet personal computer (PC) possessed by each user.

The user terminal 200 may include a mode selection part 210, a control part 220, an analysis part 230, and a display part 240.

The mode selection part 210 may provide a plurality of skin measurement modes to the user through the display part 240. The mode selection part 210 may provide a selection result of the measurement mode to the control part 220. Here, the measurement mode provided by the mode selection part 210 may include a transepidermal water loss measurement mode, a stratum corneum hydration measurement mode, and a skin image measurement mode.

The control part 220 may generate a control signal for controlling an operation of the skin condition measuring apparatus 100 based on the selection result provided by the mode selection part 210. The control part 220 may transmit the generated control signal to the skin condition measuring apparatus 100.

For example, when the transepidermal water loss measurement mode is selected in the mode selection part 210, the control part 220 may generate a control signal for operating the first sensor part 131 of the sensor unit 130 of the skin condition measuring apparatus 100, and transmit the control signal to the skin condition measuring apparatus 100. In addition, when the stratum corneum hydration measurement mode is selected in the mode selection part 210, the control part 220 may generate a control signal for operating the second sensor part 133 of the sensor unit 130 of the skin condition measuring apparatus 100, and transmit the control signal to the skin condition measuring apparatus 100. In addition, when the skin image measurement mode is selected in the mode selection part 210, the control part 220 may generate a control signal for operating the image capturing unit 120 of the skin condition measuring apparatus 100, and transmit the control signal to the skin condition measuring apparatus 100.

Meanwhile, when a plurality of measurement modes are selected in the mode selection part 210, the control part 220 may generate a control signal according to each measurement mode, and transmit the control signal to the skin condition measuring apparatus 100. In this case, the control part 220 may generate and transmit a control signal for operating the sensor unit 130 and the image capturing unit 120 of the skin condition measuring apparatus 100 together with each other or generate and transmit a control signal for sequentially operating the sensor unit 130 and the image capturing unit 120.

The analysis part 230 may analyze the user's skin condition from the measurement results of the skin condition, for example, the transepidermal water loss amount, the skin water content, or the skin image, transmitted from the skin condition measuring apparatus 100.

For example, the analysis part 230 may analyze water permeability of the user's skin barrier layer based on the transepidermal water loss amount. In addition, the analysis part 230 may analyze water of a stratum corneum of the user's skin based on the skin water content. In addition, the analysis part 230 may analyze a sebum secretion amount, a wrinkle formation degree, a colored area of a melanin pigment, or areas of pores of the user based on the skin image.

The display part 240 may display the measurement results transmitted from the skin condition measuring apparatus 100 or analysis results of the skin condition by the analysis part 230 to the user.

Meanwhile, although not illustrated in the drawings, the user terminal 200 may further include a storage part (not illustrated) capable of storing the analysis results of the skin condition by the analysis part 230.

The skin care server 300 may be connected to one or more user terminals 200 through a wired/wireless network (not illustrated). The skin care server 300 may comprehensively determine the user's skin condition by synthesizing the analysis results of the skin condition transmitted from the user terminal 200. The skin care server 300 may include a skin condition determining module 310 and a database 320.

The skin condition determining module 310 may determine the user's skin condition based on the analysis results of the skin condition transmitted from the user terminal 200. The skin condition determining module 310 may collect the analysis results of the user's skin condition for a predetermined period and comprehensively review the collected analysis results to determine the skin condition for each user.

The database 320 may store a determination result of the user's skin condition by the skin condition determining module 310. In addition, the database 320 may transmit the determination result to the user terminal 200 to allow the user terminal 200 to display the determination result to the user.

As described above, the present disclosure may complexly measure the transepidermal water loss, the stratum corneum hydration, and the skin image through one skin condition measuring apparatus 100, and comprehensively analyze various skin conditions for the user's skin, for example, the water permeability of the skin barrier layer based on the transepidermal water loss, a water content of the stratum corneum based on the stratum corneum hydration, and the sebum secretion amount, the wrinkle formation degree, the colored area of the melanin pigment, or the areas of pores of the user based on the skin image from the measurement results.

Accordingly, the present disclosure may solve inconvenience in measuring the user's skin condition and improve efficiency of measurement, determination, and care of the user's skin condition by measuring and analyzing various skin condition indexes of the user in a non-face-to-face manner through one skin condition measuring apparatus 100.

Meanwhile, it has been described by way of example that the skin condition care system 10 according to the present disclosure analyzes various skin conditions of the user by bringing the skin condition measuring apparatus 100 into contact with the skin of a user's face or body or arms and legs and measuring the transepidermal water lost, the stratum corneum hydration, or the skin image of the skin.

However, the present disclosure is not limited thereto, and the skin condition care system 10 may be used to analyze a user's scalp condition through various measurement values for a user's scalp through the skin condition measuring apparatus 100 in a state of bringing the skin condition measuring apparatus 100 into contact with the user's scalp.

For example, the skin condition care system 10 may analyze the scalp condition from measurement results, that is, a water loss amount or a water content of the scalp, of the sensor unit 130 of the skin condition measuring apparatus 100. In this case, the skin condition care system 10 may analyze a degree of damage to a stratum corneum of the scalp from the measurement results of the sensor unit 130.

In addition, the skin condition care system 10 may analyze scalp and hair conditions from a captured image, that is, a scalp image or a hair image, of the image capturing unit 120 of the skin condition measuring apparatus 100. In this case, the skin condition care system 10 may analyze a scalp keratin area, a scalp sensitivity, a hair density, or a hair thickness from the captured image of the image capturing unit 120.

As such, the skin condition care system 10 according to the present disclosure may be utilized to determine whether or not the user is losing his/her hair or a type of hair loss by measuring and analyzing the user's scalp condition or hair condition through the skin condition measuring apparatus 100.

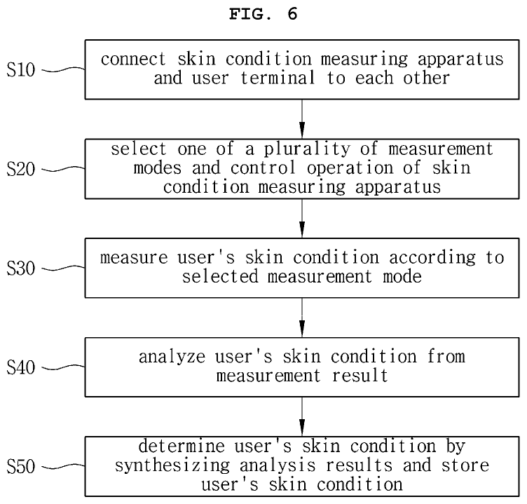

FIG. 6 is a view illustrating a skin condition care method according to an embodiment of the present disclosure.

First, the user may establish connection between the skin condition measuring apparatus 100 and the user terminal 200 to each other through his/her user terminal 200 (S10).

For example, the user terminal 200 and the skin condition measuring apparatus 100 may be connected to each other through Bluetooth wireless communication. Accordingly, the user terminal 200 may determine whether or not the skin condition measuring apparatus 100 exists within its Bluetooth communication range, and may establish communication with the corresponding skin condition measuring apparatus 100 accordingly.

Next, the user terminal 200 may provide a plurality of skin condition measurement modes to the user through the mode selection part 210, and control an operation of the skin condition measuring apparatus 100 through the control part 220 based on one or more measurement modes selected by the user (S20).

Here, the measurement mode may include a transepidermal water loss measurement mode, a stratum corneum hydration measurement mode, and a skin image measurement mode. One or more of these measurement modes maybe selected, and the control part 220 may generate a control signal for controlling an operation of the skin condition measuring apparatus 100 according to a selection result and transmit the control signal to the skin condition measuring apparatus 100.

Next, the skin condition measuring apparatus 100 may measure the user's skin condition by controlling an operation of the sensor unit 130 or the image capturing unit 120 according to the control signal transmitted from the user terminal 200 (S30).

When the transepidermal water loss measurement mode is selected in the user terminal 200 and a corresponding control signal is transmitted to the skin condition measuring apparatus 100 through the control part 220, the skin condition measuring apparatus 100 may measure a water amount lost from the user's skin, that is, the transepidermal water loss amount, through the first sensor part 131 of the sensor unit 130.

In addition, when the stratum corneum hydration measurement mode is selected in the user terminal 200 and a corresponding control signal is transmitted to the skin condition measuring apparatus 100 through the control part 220, the skin condition measuring apparatus 100 may measure the water content of the user's skin through the second sensor part 133 of the sensor unit 130.

In addition, when the skin image measurement mode is selected in the user terminal 200 and a corresponding control signal is transmitted to the skin condition measuring apparatus 100 through the control part 220, the skin condition measuring apparatus 100 may capture the image of the surface of the user's skin through the image capturing unit 120 and output the skin image.

In this case, the image capturing unit 120 may irradiate the user's skin with at least one of the white light, the polarized light, and the ultraviolet light according to the control signal, capture an image of the user's skin irradiated with the light, and output the skin image.

Then, the skin condition measuring apparatus 100 may transmit the measurement result of the sensor unit 130 or the skin image of the image capturing unit 120 to the user terminal 200. The user terminal 200 may analyze the user's skin condition based on the measurement result or the skin image (S40).

The analysis part 230 of the user terminal 200 may analyze the water permeability of the user's skin barrier layer based on the transepidermal water loss amount, that is, the transepidermal water loss, transmitted from the skin condition measuring apparatus 100.

In addition, the analysis part 230 may analyze a water content of a stratum corneum of the user's skin based on the water content, that is, the stratum corneum hydration, transmitted from the skin condition measuring apparatus 100.

In addition, the analysis part 230 may analyze the sebum secretion amount, the wrinkle formation degree, the colored area of the pigment, or the areas of pores from the user's skin based on the skin image transmitted from the skin condition measuring apparatus 100.

Here, when the image capturing unit 120 of the skin condition measuring apparatus 100 captures an image of the user's skin by irradiating the user's skin with the white light, the analysis part 230 may analyze a condition of a skin surface such as wrinkles, pores, and dead skin cells of the user's skin from a skin image by the white light, for example, a first skin image.

In addition, when the image capturing unit 120 of the skin condition measuring apparatus 100 captures an image of the user's skin by irradiating the user's skin with the polarized light, the analysis part 230 may analyze a skin tone such as a colored area of a pigment of the user's skin from a skin image by the polarized light, for example, a second skin image.

In addition, when the image capturing unit 120 of the skin condition measuring apparatus 100 captures an image of the user's skin by irradiating the user's skin with the ultraviolet light, the analysis part 230 may analyze skin damage due to a skin trouble such as acne or skin damage due to ultraviolet rays or the like from a skin image by the ultraviolet light, for example, a third skin image.

The user terminal 200 may display the analysis results of the user's skin condition described above to the user through the display part 240 and at the same time, transmit the analysis results to the skin care server 300 through a network. The skin care server 300 may determine the user's skin condition by synthesizing the analysis results, and store a determination result for each user (S50).

As such, in the skin condition care method according to the present embodiment, it is possible to comprehensively analyze the users'skin conditions such as the water permeability of the skin barrier layer, the water content of the stratum corneum, and the sebum secretion amount, the wrinkle formation degree, the colored area of the melanin pigment, or the areas of pores of the user from the transepidermal water loss, the stratum corneum hydration, and the skin image for the user's skin measured through one skin condition measuring apparatus 100.

Accordingly, the present disclosure may solve inconvenience in measuring the user's skin condition and improve efficiency of measurement, determination, and care of the user's skin condition by measuring and analyzing various skin condition indexes of the user in a non-face-to-face manner through one skin condition measuring apparatus 100.

Meanwhile, the skin condition care method described above may be implemented as a program or an application for performing the skin condition care method stored and driven in a computer-readable recording medium. Accordingly, the user terminal 200 or the skin care server 300 according to the present disclosure may be loaded with the program or the application for performing the skin condition care method described above, and may perform an operation such as comprehensive analysis and determination of the user's skin condition by driving the program or the application.

According to the present disclosure, it is it is possible to comprehensively analyze user's skin condition such as the water permeability of the skin barrier layer, the water content of the stratum corneum, and the sebum secretion amount, the wrinkle formation degree, the colored area of the melanin pigment, or the areas of pores of the user from the transepidermal water loss, the stratum corneum hydration, and the skin image for the user's skin measured through one skin condition measuring apparatus.

Accordingly, the present disclosure may solve inconvenience in measuring the user's skin condition and improve efficiency of measurement, determination, and care of the user's skin condition by measuring and analyzing various skin condition indexes of the user in a non-face-to-face manner through one skin condition measuring apparatus.

The embodiments of the present disclosure have been described hereinabove, but those skilled in the art may variously modify and alter the present disclosure by adding, changing or deleting components without departing from the spirit and scope of the present disclosure defined in the claims, and it is to be considered that these modifications and alterations fall in the scope of the present disclosure.

In addition, the terms described above are defined in consideration of functions in the present disclosure, and may be changed by the intention of users or operators or practice. Therefore, these terms should be defined on the basis of the contents throughout the present specification.

What is claimed is:

1. A portable skin condition measuring apparatus for measuring a skin condition of a user's face, body, or scalp, comprising:
a main body having a skin measurement region formed therein;
a sensor unit disposed inside the main body and measuring the skin condition through the skin measurement region;
an image capturing unit disposed inside the main body and configured to capture an image of a user's skin through the skin measurement region; and
a control unit configured to control an operation of at least one of the sensor unit and the image capturing unit based on a control signal transmitted from a user terminal, and configured to transmit at least one of measurement results of the sensor unit and the image of the user's skin to the user terminal,
wherein the skin measurement region is partitioned into a first region and a second region by a partitioning part, and the first region has a shape in which one surface thereof in contact with the user's skin is opened.

2. The portable skin condition measuring apparatus of claim 1, wherein the sensor unit comprises:
a first sensor part disposed in the first region and configured to measure a transepidermal water loss amount of a user; and
one or more second sensor parts disposed at one end of the main body, in contact with the user's skin, and configured to measure a water content of the user's skin.

3. The portable skin condition measuring apparatus of claim 1, wherein the image capturing unit comprises:
a light source part comprising a plurality of light sources irradiating to the user's skin with at least one of white light, polarized light, and ultraviolet light through the first region according to control of the control unit; and
an image capturing part disposed in the second region and configured to capture an image of a surface of the user's skin through the skin measurement region.

4. The portable skin condition measuring apparatus of claim 3, wherein the light source part further comprises a polarizing film disposed on a light emission surface of at least one light source irradiating the user's skin with the white light among the plurality of light sources.

5. The portable skin condition measuring apparatus of claim 3, wherein a light transmitting surface is formed on an inner surface of the first region so that the light emitted from the light source part is transmitted.

6. The portable skin condition measuring apparatus of claim 1, wherein the image capturing unit is configured to output a first skin image captured by the white light with which the user's skin is irradiated, a second skin image captured by the polarized light with which the user's skin is irradiated, and a third skin image captured by the ultraviolet light with which the user's skin is irradiated as the skin image.

7. A skin condition care system comprising:
the skin condition measuring apparatus of claim 1;
a user terminal configured to control an operation of the skin condition measuring apparatus based on one or more measurement modes selected from the group consisting of a plurality of skin measurement modes, to receive at least one of a transepidermal water loss amount, a skin water content, and a skin image for the user's skin, and to analyze the user's skin condition; and
a skin care server configured to receive an analysis result from the user terminal, to determine the user's skin condition, and to store a determination result,
wherein the skin measurement mode comprises a transepidermal water loss measurement mode, a stratum corneum hydration measurement mode, or a skin image measurement mode.

8. The skin condition care system of claim 7, wherein the user terminal comprises a control part configured to generate a control signal transmitted to the skin condition measuring apparatus based on the selected one or more measurement modes; and
the control part is configured to:
generate a control signal for operating a first sensor part of the sensor unit of the skin condition measuring apparatus when the transepidermal water loss measurement mode is selected;
generate a control signal for operating a second sensor part of the sensor unit of the skin condition measuring apparatus when the stratum corneum hydration measurement mode is selected; and
generate a control signal for operating the image capturing unit of the skin condition measuring apparatus when the skin image measurement mode is selected.

9. The skin condition care system of claim 7, wherein the user terminal comprises an analysis unit configured to analyze the user's skin condition from at least one of the transepidermal water loss amount, the skin water content, and the skin image provided from the skin condition measuring apparatus; and
the analysis unit is configured to analyze water permeability of a user's skin barrier layer from the transepidermal water loss amount, analyzes a water content of a stratum corneum of the user's skin from the skin water content, and analyze a sebum secretion amount, a wrinkle formation degree, a colored area of a melanin pigment, or areas of pores of a user from the skin image.

10. The skin condition care system of claim 7, wherein the skin condition measuring apparatus is configured to output a first skin image captured by white light with which the user's skin is irradiated, a second skin image captured by polarized light with which the user's skin is irradiated, and a third skin image captured by ultraviolet light with which the user's skin is irradiated as the skin image.

11. A skin condition care method of measuring and managing a skin condition of a user's face, body, or scalp using a skin condition care system in which a skin condition measuring apparatus, a user terminal and a skin care server are connected to each other through a wired/wireless network, the method comprising:
generating a control signal for controlling an operation of the skin condition measuring apparatus based on at least one of a transepidermal water loss measurement mode, a stratum corneum hydration measurement mode, or a skin image measurement mode selected through the user terminal;
measuring a user's skin condition through the skin condition measuring apparatus controlled by the control signal;
receiving at least one of measurement results and a skin image from the skin condition measuring apparatus and analyzing the user's skin condition; and
determining the user's skin condition by synthesizing the analysis results, and storing a determination result.

12. The skin condition care method of claim 11, wherein the generating of the control signal comprises:
generating a control signal for operating a first sensor part of the sensor unit of the skin condition measuring apparatus when the transepidermal water loss measurement mode is selected;
generating a control signal for operating a second sensor part of the sensor unit of the skin condition measuring apparatus when the stratum corneum hydration measurement mode is selected; and
generating a control signal for operating the image capturing unit of the skin condition measuring apparatus when the skin image measurement mode is selected.

13. The skin condition care method of claim 11, wherein the analyzing of the user's skin condition comprises:
analyzing water permeability of a skin barrier layer based on a measurement result for a transepidermal water loss amount provided from the skin condition measuring apparatus;
analyzing a water content of a stratum corneum of a user's skin based on a measurement result for a skin water content provided from the skin condition measuring apparatus; and
analyzing a sebum secretion amount, a wrinkle formation degree, a colored area of a melanin pigment, or areas of pores of a user from the skin image based on the skin image.

14. The skin condition care method of claim 11, wherein the analyzing of the user's skin condition comprises:
receiving one of a first skin image by white light with which the user's skin is irradiated, a second skin image by polarized light with which the user's skin is irradiated, and a third skin image by ultraviolet light with which the user's skin is irradiated; and
analyzing a condition of a skin surface comprising wrinkles, pores, and dead skin cells of a user's skin from the first skin image, analyzing a skin tone comprising a colored area of a pigment of the user's skin from the second skin image, and analyzing skin damage due to a skin trouble comprising acne or skin damage due to ultraviolet rays from the third skin image.
